(19) United States
(12) Patent Application Publication
(10) Pub. No.: US 2025/0318744 A1
(43) Pub. Date: Oct. 16, 2025

(54) UNIVERSAL WIRELESS RADIO FREQUENCY COIL SYSTEM AND IMAGING METHOD FOR MAGNETIC RESONANCE IMAGING

(71) Applicant: SHENZHEN INSTITUTES OF ADVANCED TECHNOLOGY CHINESE ACADEMY OF SCIENCES, Shenzhen (CN)

(72) Inventors: Ye LI, Shenzhen (CN); Zhiguang MO, Shenzhen (CN); Hairong ZHENG, Shenzhen (CN)

(73) Assignee: SHENZHEN INSTITUTES OF ADVANCED TECHNOLOGY CHINESE ACADEMY OF SCIENCES, Shenzhen (CN)

(21) Appl. No.: 19/063,760

(22) Filed: Feb. 26, 2025

Related U.S. Application Data
(63) Continuation of application No. PCT/CN2024/087556, filed on Apr. 12, 2024.

(51) Int. Cl. A61B 5/055; A61B 5/00; G01R 33/34

(52) U.S. Cl. CPC A61B 5/055; G01R 33/34084; A61B 5/742

(57) ABSTRACT

Disclosed are universal wireless radio frequency coil system and imaging method for magnetic resonance imaging. The system includes a transmitting coil, a universal pickup coil and a wireless radio frequency coil. The universal pickup coil and the wireless radio frequency coil form wireless radio frequency coil assembly, and the transmitting coil is for transmitting a magnetic resonance signal aiming at a target part to be imaged so that the target part is excited to generate an electromagnetic signal; the wireless radio frequency coil consists of a plurality of wireless radio frequency coil units and is for amplifying the electromagnetic signals and transmitting the amplified electromagnetic signals to the universal pickup coil in a magnetic coupling mode; and the universal pickup coil encircles the wireless radio frequency coil and a target part where the wireless radio frequency coil is worn in a manner covering a range of 360 degrees.

### CROSS-REFERENCE TO RELATED APPLICATIONS

This application is a continuation application of International Application No. PCT/CN2024/087556, filed on Apr. 12, 2024, the entire contents of which are incorporated herein by reference.

### TECHNICAL FIELD

The present application relates to the field of magnetic resonance imaging technology, and more particularly, to a universal wireless radio frequency coil system and imaging method for magnetic resonance imaging.

### BACKGROUND

Magnetic resonance imaging (MRI) has become an important means of soft tissue imaging because of its advantages of non-invasive, non-radiation, high-resolution, high-contrast and arbitrary orientation cross-section imaging. The process of magnetic resonance signal acquisition mainly includes radio frequency excitation stage and radio frequency reception stage. Referring to the schematic diagram of the traditional wired coil in FIG. 1, in the radio frequency excitation stage, the magnetic resonance imaging system sends magnetic resonance signals to the human tissue in the wired coil 11 through the transmitting coil 3 (the transmitting coil 3 is a body coil, and the body coil can be used as a transmitting coil or a receiving coil). In the radio frequency reception stage, the excited tissue sends an electromagnetic signal to the surrounding space, and the electromagnetic signal is received by the receiving coil 11 and fed back to the imaging system, and the signal acquisition process is completed. As the electromagnetic signal is very weak, the performance of the receiving coil is critical and largely determines the final image quality.

The performance of the receiving coil is mainly reflected in the sensitivity and parallel imaging capability. High sensitivity means that weak signals can be resolved, and high parallel imaging capability determines that the magnetic resonance system can perform faster imaging in hardware. In order to obtain the best possible image quality, the existing technology typically customizes a dedicated wired receiving coil 11 for different body parts, such as head coil, knee coil and abdomen coil. When performing magnetic resonance imaging of different parts, doctors need to change different wired coils back and forth. In addition, the traditional receiving coils are composed of a large number of resonant units, matching/tuning circuits, pre-amplifier circuits and transmission cables with trappers. Such structures result in coils that are very bulky, such as common head, knee, and abdominal coils that weigh several kilograms. The repeated replacement of such heavy coils and cables brings physical burden to the doctor's work. When the doctor replaces and carries the wired coils, the patient can only wait by the side, which greatly affects the efficiency of the examination. In addition, due to the bulkiness of the existing wired abdominal coil, the patient needs to bear the compression of the abdominal coil all the time during the abdominal imaging, which not only has potential safety hazards to the injured or weak patients, but also greatly affects the comfort, and is easy to cause the patient to be uneasy and twist, resulting in image artifacts.

Through analysis, the current wired or wireless radio frequency coil mainly has the following defects.

Firstly, in the prior art, a spine coil is used as a pickup coil. The spine coil is usually an overlapping multi-channel coil array, which is tiled on the hospital bed. Because the spine coil is only distributed under the wireless radio frequency coil, the signals in other directions will be lost, thus affecting the overall sensitivity. Since the parallel imaging capability of a wireless radio frequency coil depends on the number of channels of a pickup coil and the spatial angle covered by the pickup coil, and a spine coil (arranged on a hospital bed) is only distributed below the wireless radio frequency coil, the spatial angle covered by the spine coil is limited relative to an imaging target area and the wireless radio frequency coil, thereby affecting the parallel imaging capability.

Secondly, in the prior art, a body coil is used as a pickup coil. The body coil is typically a bird cage coil integrated within a magnet because the sensitivity of the bird cage coil is typically weaker than array of surface coils of the same size. However, the bird cage coil is usually too far away from the wireless radio frequency coil, and the use of the bird cage coil as a pickup coil reduces the sensitivity. In addition, the bird cage coil has only two channels, while the parallel imaging capability of a wireless radio frequency coil system depends on the number of channels of the pickup coil, and the more the number of channels, the stronger the parallel imaging capability, therefore, the scheme of using a body coil (bird cage coil) also affects the parallel imaging capability.

### SUMMARY

The purpose of the present application is to overcome the shortcomings of the prior art and provide a universal wireless radio frequency coil system and an imaging method for magnetic resonance imaging.

According to the first aspect of the present application, there is provided a universal wireless radio frequency coil system for magnetic resonance imaging, comprising a transmitting coil, a universal pickup coil and a wireless radio frequency coil, wherein the universal pickup coil and the wireless radio frequency coil form a wireless radio frequency coil assembly, and the transmitting coil is used for transmitting a magnetic resonance signal aiming at a target part to be imaged so that the target part is excited to generate an electromagnetic signal; the wireless radio frequency coil consists of a plurality of wireless radio frequency coil units and is used for amplifying the electromagnetic signals and transmitting the amplified electromagnetic signals to the universal pickup coil in a magnetic coupling mode; and the universal pickup coil encircles the wireless radio frequency coil and the target part where the wireless radio frequency coil is worn in a manner covering a range of 360 degrees.

According to the second aspect of the present application, there is provided an imaging method, comprising:
performing magnetic resonance scanning on a target part by using the universal wireless radio frequency coil system provided; and
acquiring and displaying an imaging result of the magnetic resonance scanning.

Compared with the prior art, the application has the advantages that the universal wireless radio frequency coil system for magnetic resonance imaging is designed, the sensitivity and the parallel imaging performance of the magnetic resonance imaging can be improved, and the system is particularly suitable for high-sensitivity and high-parallel imaging of different organs of a human body.

Other features and advantages of the present application will become apparent from the following detailed description of exemplary embodiments of the application with reference to the figures.

### BRIEF DESCRIPTION OF THE DRAWINGS

The figures, which are incorporated in and constitute a part of the specification, illustrate embodiments of the application and, together with the description, serve to explain the principles of the application. In the drawings:

FIG. 1 is a schematic diagram of a wired radio frequency coil in the prior art;

FIG. 2 is a front view of a universal wireless radio frequency coil system for magnetic resonance imaging according to one embodiment of the present application;

FIG. 3 is a schematic diagram of cross-sectional view 1 of the universal wireless radio frequency coil system of FIG. 2;

FIG. 4 is a schematic diagram of cross-sectional view 2 of the universal wireless radio frequency coil system of FIG. 2;

FIG. 5 is a flow diagram of an imaging method based on a universal wireless radio frequency coil system according to an embodiment of the present application;

FIG. 6 is a flow diagram of an imaging method of a universal wired radio frequency coil system;

FIG. 7 is a schematic diagram of a wireless radio frequency coil unit according to an embodiment of the present application; and

FIG. 8 is a schematic diagram of a wireless radio frequency coil array according to one embodiment of the present application.

### DETAILED DESCRIPTION OF THE EMBODIMENTS

Various exemplary embodiments of the present application will now be described in detail with reference to the figures. It should be noted that the relative arrangement of parts and steps, the numerical expressions, and the numerical values set forth in these embodiments do not limit the scope of the application unless specifically stated otherwise.

The following description of at least one exemplary embodiment is merely illustrative in nature and is in no way intended to limit the application, its application, or uses.

Techniques, methods, and devices known to those of ordinary skill in the pertinent art may not be discussed in detail, but should be considered part of the specification, where appropriate.

In all instances shown and discussed herein, any particular value is to be construed as merely illustrative and not restrictive. Thus, other examples of example embodiments may have different values.

It should be noted that like reference numbers and letters refer to like items in the following figures, and therefore, once an item is defined in one figure, it needs not be further discussed in subsequent figures.

The application provides a universal wireless radio frequency coil system for magnetic resonance imaging and a corresponding imaging method. In general, the universal wireless radio frequency coil system takes a surface coil array as a pickup coil and is formed by matching wireless radio frequency coils at different parts. Compared with the existing scheme, the imaging method based on the universal wireless radio frequency coil system has higher imaging sensitivity and stronger parallel imaging capability.

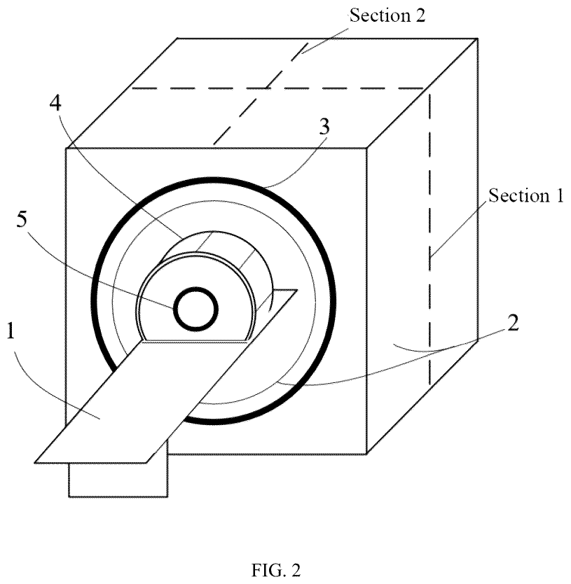

Specifically, as shown in FIG. 2, the provided universal wireless radio frequency coil system mainly comprises a transmitting coil 3, a universal pickup coil 4 and a wireless radio frequency coil 5, wherein the wireless radio frequency coil 5 can be designed to adapt to different imaging parts, that is, the wireless radio frequency coil 5 with a suitable form is equipped for different human body parts. The universal pickup coil 4 and the wireless radio frequency coil 5 constitute a radio frequency coil assembly. FIG. 2 also illustrates a magnetic resonance hospital bed 1 and a magnet 2.

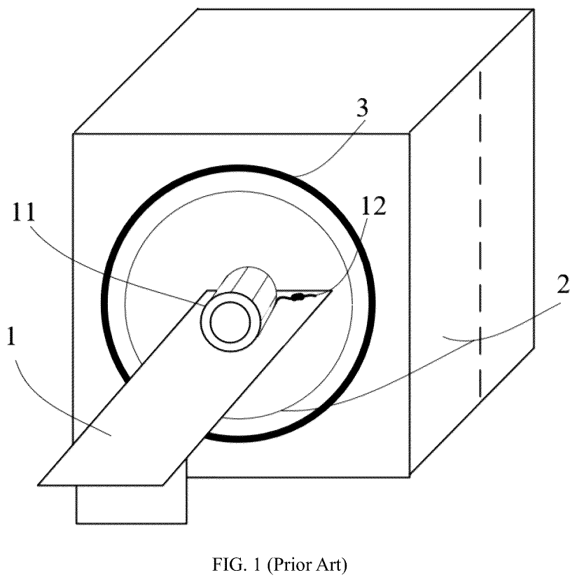

In the present application, the wireless radio frequency coil 5 is not physically connected to the main body of the MRI apparatus, and the combination of the universal pickup coil 4 and the wireless radio frequency coil 5 is used instead of the wired receiving coil 11 in FIG. 1 in the prior art to achieve higher imaging quality.

In one embodiment, the wireless radio frequency coil assembly comprises a universal pickup coil 4 and a plurality of different portions of the wireless radio frequency coil 5. When in use, the universal pickup coil 4 surrounds the wireless radio frequency coil 5 and the imaging object in the radio frequency coil by one circle, covering a range of 360 degrees. This omni-directional spatial angle coverage for the imaging object or the imaging target part improves the parallel imaging capability. The wireless radio frequency coil 5 may send, receive, or both transmit and receive radio frequency signals of the imaging target part. In this application, the wireless radio frequency coil working in the radio frequency receiving stage is taken as an example to illustrate.

In one embodiment, in order to meet the high parallel imaging performance, the universal pickup coil 4 is constituted by a wired surface coil array for magnetic resonance imaging to enable acquisition of magnetic resonance signals, and is set to have 6 or more channels. In addition, the aperture of the pickup coil 4 is set to be large enough to accommodate a person lying therein for imaging different target parts.

In one embodiment, the wireless radio frequency coil 5 is configured to operate in a detuned state or resonant state. For example, in a detuned state during the transmitting stage of the magnetic resonance system and in a resonant state during the receiving stage of the magnetic resonance system. The wireless radio frequency coil 5 may be constituted by a single or a plurality of resonant units capable of realizing electromagnetic resonance within the resonance frequency of the magnetic resonance system of ±100 MHz.

The transmitting coil 3 is used for transmitting a magnetic resonance signal to a target part to be imaged so that the target part is excited to generate an electromagnetic signal. The transmitting coil 3 may be implemented as a body coil or other type of coil.

It shall be noted that, the wireless radio frequency coil assembly provided by the present application mainly works in the receiving stage, therefore, in the transmitting stage, the wireless radio frequency coil should be in the parallel resonance state, during the parallel resonant state, the wireless radio frequency coil is equivalent to an open circuit, therefore, when the electromagnetic excitation occurs in the transmitting stage, the wireless radio frequency coil does not generate an induced current, thereby avoiding interference with the magnetic field during the transmitting stage.

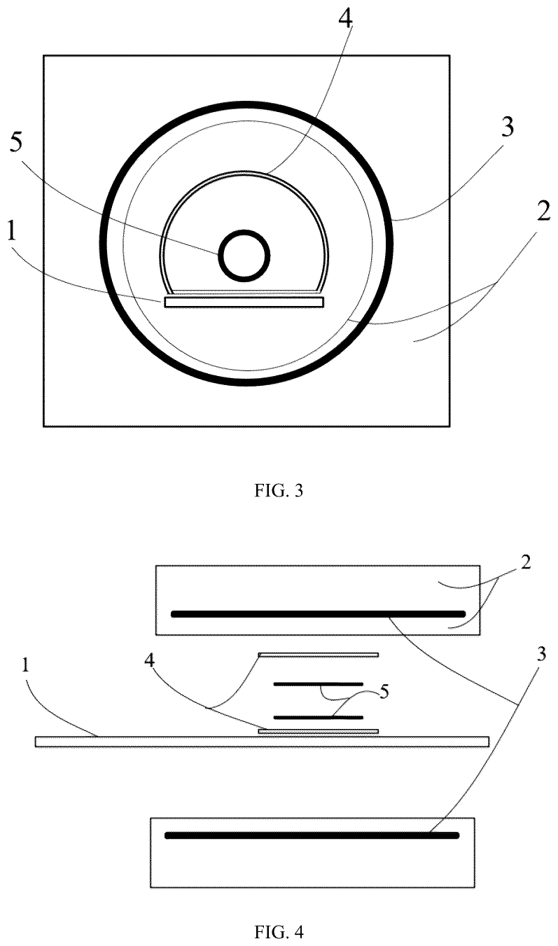

For the universal radio frequency coil system in the working state, the relative positional relationship among the transmitting coil 3 (taking the body coil as an example), the universal pickup coil 4, and the wireless radio frequency coil 5 is shown in FIG. 2, FIG. 3 and FIG. 4.

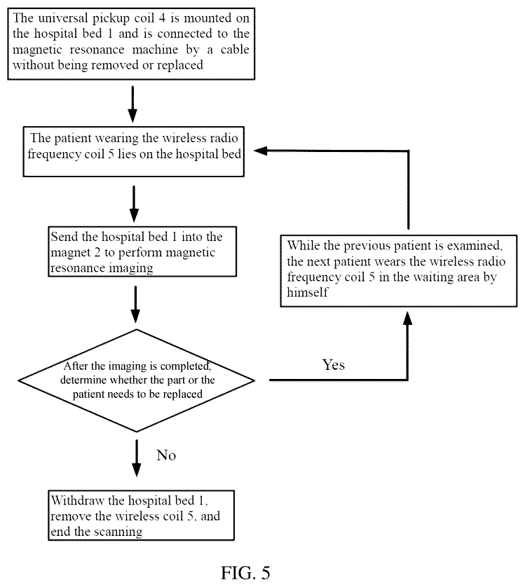

FIG. 5 is a flow diagram of an imaging method based on the universal wireless radio frequency coil system provided by the present application, the imaging method comprising the following steps.

S1, the universal pickup coil 4 is placed on the magnetic resonance hospital bed 1 and connected to the magnetic resonance machine through a cable, and the universal pickup coil 4 does not need to be removed or replaced when the scanning part and the patient are replaced.

S2, the patient wearing the wireless radio frequency coil 5 lies on the hospital bed 1.

S3, sending the hospital bed 1 into the magnet 2 to perform magnetic resonance imaging.

S4, after the imaging is completed, it is judged whether the part or the patient needs to be replaced.

If the part or the patient needs to be replaced, Step S5 is executed, otherwise, Step S6 is executed.

S5, while the previous patient is examined, the next patient wears the wireless radio frequency coil 5 in the waiting area by himself.

For example, when the previous patient is receiving examination, the next patient can wear the wireless radio frequency coil 5 of the corresponding part in the waiting area by himself, and then the part to be tested and the wireless radio frequency coil 5 are put into the universal pickup coil 4, and the hospital bed 1 is sent into the magnet 2 to start magnetic resonance scanning.

S6, withdraw the hospital bed 1, remove the wireless radio frequency coil 5, and end the scanning.

In general, when the magnetic resonance imaging system works, a magnetic resonance signal is transmitted through the body coil 3, the tissue at the part of the patient to be imaged is excited to transmit an electromagnetic signal, the electromagnetic signal is amplified when passing through the wireless radio frequency coil 5, and the amplified signal is transmitted to the universal pickup coil 4 in a magnetic coupling manner to complete signal acquisition.

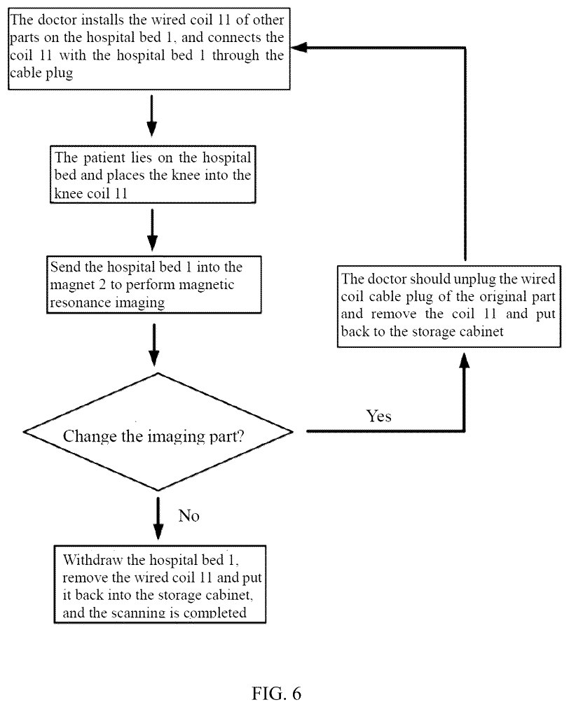

For comparison with the prior art, FIG. 6 illustrates an imaging process based on the wired coil shown in FIG. 1. When the tissue of the part of the patient to be imaged is excited to emit an electromagnetic signal, the electromagnetic signal is directly collected by the wired coil. On the premise that the imaging part needs to be replaced, the doctor should unplug the wired coil cable plug of the original part and remove the coil 11. Therefore, compared with the existing technology, the method has the advantages that the carrying process of the wired coil is omitted, the next patient can automatically wear the wireless coil in the waiting area while the previous patient receives the examination, and the patient only needs to walk into the magnet room and lie on the hospital bed during the examination, so that the process of the magnetic resonance examination is simplified, and the efficiency is improved. In addition, because the wireless radio frequency coil adopted by the application does not need the structures such as a pre-amplifier circuit, a transmission cable with a trapper and the like, the wireless radio frequency coil is very light and thin, and the safety and the use comfort of a patient are improved.

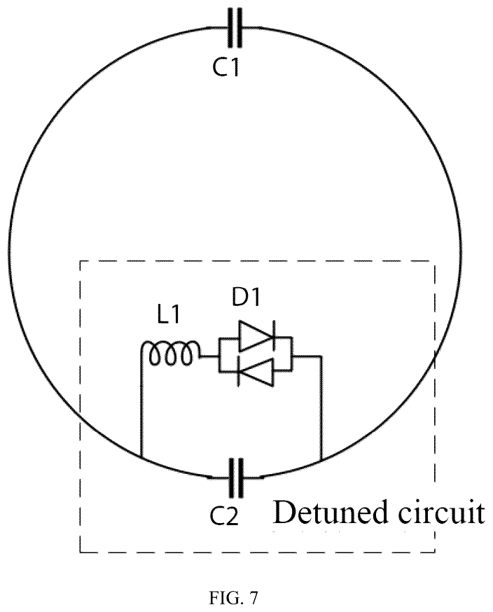

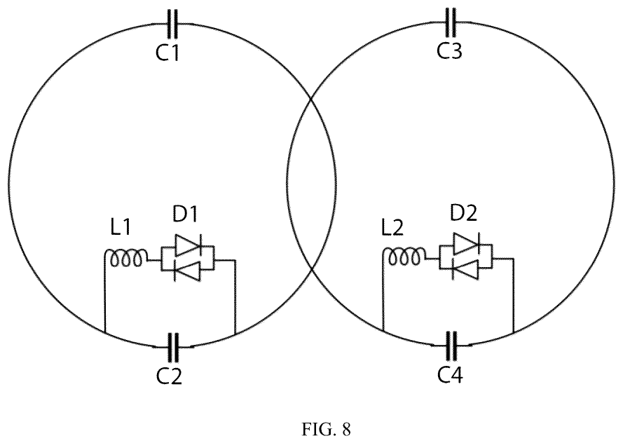

In actual use, that wireless radio frequency coil may comprise a plurality of wireless radio frequency coil units. FIG. 7 is a schematic diagram of a wireless radio frequency coil unit. Each wireless radio frequency coil unit comprises a capacitor C1, a capacitor C2, a bidirectional diode D1, and an inductor L1. The wireless radio frequency coil unit has a detuned circuit and a resonance circuit. The detuned circuit consists of an inductor L1, a capacitor C2, and a bidirectional diode D1. In the signal excitation stage, the diode is turned on, and the circuit is in the detuned state (or parallel resonant state). In the receiving stage, the diode is not turned on and the circuit is in resonant state. The wireless radio frequency coil is detuned within the operating frequency of the system of ±100 MHZ, and when the wireless radio frequency coil is in the signal receiving stage, the diode is not turned on, and the wireless radio frequency coil is resonated within the working frequency of the system of ±100 MHz. A wireless radio frequency coil array composed of a plurality of wireless radio frequency units is shown in FIG. 8.

It should be noted that in the transmitting stage, the detuned of the wireless coil within the system operating frequency of ±100 MHz reduces the interference of the wireless radio frequency coil on the transmitting field in the system transmitting stage; In the receiving stage, the wireless radio frequency coil resonates within the system operating frequency of #100 MHz to amplify the signal, so that the high sensitivity of the receiving coil can be realized.

It should be understood that those skilled in the art can make appropriate changes or modifications to the above embodiments without departing from the spirit and scope of the present application. For example, the present application is not limited to use for human body imaging, but is also applicable to animal imaging. The universal pickup coil is not limited to be placed on the hospital bed, but is also suitable to be integrated in the magnet. In addition, the universal pickup coil is not limited to the pattern shown in the figures, and other surface coil arrays that function as signal pickups may also be used. The resonant unit structure involved is not limited to a circular shape, but is also applicable to other shapes capable of satisfying the detuned and resonance requirements. In addition, the wireless radio frequency coil is not limited to a resonant unit structure, and is also applicable to an array structure meeting detuned and resonance conditions.

In order to further verify the effect of the application, experimental verification is carried out, and the experiment shows that the universal wireless radio frequency coil system designed by the application not only can obtain higher sensitivity compared with a commercial knee coil, but also has higher parallel imaging capability compared with the existing commercial coil, and is a feasible imaging method.

In summary, the present application has the following advantages over the prior technology.

Firstly, compared with the traditional wired coil, the wireless radio frequency coil has the characteristics of lightness, thinness and the like, so that the comfort of a patient and the physical burden of a doctor are improved.

Secondly, the pickup coil of the application can be formed by adopting a wired surface coil array to realize the acquisition of magnetic resonance signals, and more than six channels are arranged to meet the requirement of high parallel imaging performance.

Thirdly, the pickup coil of the application can be fixed in the hospital bed or the aperture of the magnet to serve as the pickup coil of all the wireless radio frequency coils, and when the scanning part is replaced, the wired coil does not need to be plugged, and only the wireless coil of the corresponding scanning part is replaced, so that the use comfort of the patient is improved, the workload of the doctor is reduced, the examination process is simplified, and the efficiency of the magnetic resonance examination is improved. In addition, the application can use a multi-channel overlapping coil array as a pickup coil, and has higher imaging sensitivity and stronger parallel imaging capability compared with the existing wireless radio frequency coil scheme.

Lastly, because the multi-channel surface coil array is used as a universal pickup coil, the application has more channels, better imaging sensitivity and higher parallel imaging capability compared with the existing scheme of using a bird cage coil as a pickup coil or using a body coil.

The present application may be a system, method, and/or computer program product. The computer program product may include a computer readable storage medium that is uploaded with computer readable program instructions for causing a processor to implement various aspects of the present application.

A computer readable storage medium may be a tangible device that may hold and store instructions for use by an instruction execution device. A computer readable storage medium may be, for example but not limited to, an electronic storage device, a magnetic storage device, an optical storage device, an electromagnetic storage device, a semiconductor storage device, or any suitable combination of the foregoing. More specific examples (a non-exhaustive list) of the computer readable storage medium would include a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as a punch card or a raised structure in a groove on which instructions are stored, and any suitable combination of the foregoing. The computer readable storage medium used herein is not interpreted as an instantaneous signal itself, such as radio waves or other freely propagating electromagnetic waves, electromagnetic waves propagated through waveguides or other transmission media (e.g. optical pulses through fiber optic cables), or electrical signals transmitted through wires.

The computer readable program instructions described herein can be downloaded to various computing/processing devices from a computer readable storage medium, or to an external computer or external storage device over a network, such as the Internet, a local area network, a wide area network, and/or a wireless network. The network may include copper transmission cables, fiber optic transmission, wireless transmission, routers, firewalls, switches, gateway computers, and/or edge servers. A network adapter card or network interface in each computing/processing device receives computer readable program instructions from the network and forwards the computer-readable program instructions for storage in a computer readable storage medium in the respective computing/processing device.

The computer program instructions used to perform the operations of the present application may be assembly instructions, instruction set architecture (ISA) instructions, machine instructions, machine related instructions, microcode, firmware instructions, state setting data, or source or object code written in any combination of one or more programming languages, including object-oriented programming languages such as Smalltalk, C++, Python, and conventional procedural programming languages such as “C” language or similar programming languages. Computer readable program instructions can be executed entirely on the user's computer, partially on the user's computer, as a standalone software package, partially on the user's computer, partially on a remote computer, or entirely on a remote computer or server. In cases involving remote computers, the remote computer can be connected to the user's computer through any type of network, including a local area network (LAN) or a wide area network (WAN), or can be connected to an external computer (such as using an internet service provider to connect through the internet). In some embodiments, various aspects of the application are implemented by personalizing an electronic circuit, such as a programmable logic circuit, a field programmable gate array (FPGA), or a programmable logic array (PLA), with state information of computer readable program instructions that the electronic circuit may execute.

Various aspects of the present application are described herein with reference to flowchart illustrations and/or block diagrams of methods, apparatus (systems) and computer program products according to embodiments of the application. It should be understood that each box in the flowchart and/or block diagram, as well as combinations of boxes in the flowchart and/or block diagram, can be implemented by computer readable program instructions.

The computer readable program instructions may be provided to a processor of a general purpose computer, special purpose computer, or other programmable data processing apparatus to produce a machine, such that the instructions, when executed by the processor of the computer or other programmable data processing apparatus, resulting in means for implementing the functions/acts specified in the flowchart and/or block diagram block or blocks. The computer readable program instructions may also be stored in a computer readable storage medium that cause a computer, programmable data processing apparatus, and/or other devices to function in a particular manner, such that the computer readable medium storing the instructions comprises an article of manufacture, Which include instructions for implementing various aspects of the functions/acts specified in the flowchart and/or block diagram block or blocks.

The computer readable program instructions may also be loaded onto a computer, other programmable data processing apparatus, or other devices to cause a series of operational steps to be performed on the computer, other programmable data processing apparatus, or other devices, to produce a computer implemented process such that the instructions which execute on the computer, other programmable data processing apparatus, or other devices implement the function/act specified in the flowchart and/or block diagram block or blocks.

The flowchart and block diagrams in the figures illustrate the architecture, functionality, and operation of possible implementations of systems, methods and computer program products according to various embodiments of the present application. In this regard, each block in the flowchart or block diagrams may represent a module, segment, or portion of instructions, which comprise one or more executable instructions for implementing the specified logical function(s). In some alternative implementations, the functions noted in the blocks may occur out of the order noted in the figures. For example, two consecutive blocks may, in fact, be executed substantially concurrently, or they may sometimes be executed in the reverse order, depending upon the functionality involved. It also should be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions. It is well known to those skilled in the art that implementation by means of hardware, implementation by means of software, and implementation by means of a combination of software and hardware are all equivalent.

Various embodiments of the present application have been described above, and the above description is exemplary, not exhaustive, and is not limited to the disclosed embodiments. Many modifications and variations will be apparent to those of ordinary skill in the art without departing from the scope and spirit of the described embodiments. The terminology used in the application was chosen to best explain the principles of the embodiments, the practical application, or technological improvements in the marketplace, or to enable others of ordinary skill in the art to understand the embodiments disclosed herein. The scope of the application is defined by the claims.

What is claimed is:

1. A universal wireless radio frequency coil system for magnetic resonance imaging, comprising a transmitting coil, a universal pickup coil and a wireless radio frequency coil, wherein
the universal pickup coil and the wireless radio frequency coil form a wireless radio frequency coil assembly, and the transmitting coil is used for transmitting a magnetic resonance signal aiming at a target part to be imaged so that the target part is excited to generate an electromagnetic signal;
the wireless radio frequency coil consists of a plurality of wireless radio frequency coil units and is used for amplifying the electromagnetic signals and transmitting the amplified electromagnetic signals to the universal pickup coil in a magnetic coupling mode; and
the universal pickup coil encircles the wireless radio frequency coil and the target part where the wireless radio frequency coil is worn in a manner covering a range of 360 degrees.

2. The universal wireless radio frequency coil system according to claim 1, wherein the wireless radio frequency coil units comprise a first capacitor, a second capacitor, a bidirectional diode and an inductor, and the wireless radio frequency coil unit is in a detuned state at the transmitting stage and is in a resonant state at a receiving stage by controlling the bidirectional diode to be turned on or turned off.

3. The universal wireless radio frequency coil system according to claim 2, wherein in the detuned state, the radio frequency coil is configured to be detuned within ±100 MHz of the operating frequency, and in the resonant state, the radio frequency coil is configured to resonate within ±100 MHz of the operating frequency.

4. The universal wireless radio frequency coil system according to claim 1, wherein the universal pickup coil is formed by a wired surface coil array, and is configured to have more than 6 channels.

5. The universal wireless radio frequency coil system according to claim 4, wherein the universal pickup coil is fixed in a magnet aperture of a hospital bed or a magnetic resonance system, and is connected with a magnetic resonance machine through a cable.

6. The universal wireless radio frequency coil system according to claim 1, wherein the aperture of the universal pickup coil is configured to accommodate a human body therein.

7. The universal wireless radio frequency coil system according to claim 1, wherein the transmitting coil is a body coil.

8. The universal wireless radio frequency coil system according to claim 1, wherein the number of the radio frequency coils is set to one or more.

9. An imaging method, comprising steps of:
performing magnetic resonance scanning on a target part by using the universal wireless radio frequency coil system according to claim 1; and
acquiring and displaying an imaging result of the magnetic resonance scanning.

10. A non-transitory computer readable storage medium, having a computer program stored thereon, wherein the computer program, when executed by a processor, implements the steps of the method according to claim 9.
